(19) United States
(12) Patent Application Publication
(10) Pub. No.: US 2023/0290103 A1
(43) Pub. Date: Sep. 14, 2023

(54) SYSTEMS AND METHODS FOR PERFORMING PHYSIOLOGICAL MEASUREMENTS

(71) Applicants: Michael Gross, Halifax (CA); John Matthews, Halifax (CA); Jimmy Chow, Halifax (CA)

(72) Inventors: Michael Gross, Halifax (CA); John Matthews, Halifax (CA); Jimmy Chow, Halifax (CA)

(21) Appl. No.: 18/119,779

(22) Filed: Mar. 9, 2023

Related U.S. Application Data
(60) Provisional application No. 63/318,254, filed on Mar. 9, 2022.

(51) Int. Cl. G06V 10/22; G06T 7/00; G06T 7/73; G06V 40/10

(52) U.S. Cl. CPC G06V 10/225; G06T 7/0012; G06T 7/73; G06V 40/10

(57) ABSTRACT

Apparatus, systems and methods for measuring changes in the volume of a tissue of a person are described herein. The methods include applying a first marker to a portion of a tissue, the first marker having a pattern of markings thereon, each marking of the pattern of markings having an initial position; capturing an image of the pattern of markings at a first time after applying the first marker to the tissue, at least one marking of the pattern of markings having a subsequent position at the first time when the image is captured; determining a change of position of the at least one marking by comparing the subsequent position of the at least one marking in the captured image to the initial position of the at least one marking; and based on the change of position of the at least one marking, determining the physiological measurement of the tissue.

### CROSS-REFERENCE

The present application claims priority of U.S. Provisional Patent Application No. 63/318,254 filed on Mar. 9, 2022 entitled Systems and Methods for Performing Physiological Measurements, the entire contents of which are hereby incorporated by reference herein.

### TECHNICAL FIELD

The embodiments disclosed herein relate to systems and methods for performing physiological measurements, and, in particular to systems and methods of performing physiological measurements such as measuring a range of motion of a joint or measuring a change in volume of a tissue.

### BACKGROUND

There are currently various techniques available for performing physiological measurements of parts of a body.

For example, when measuring a range of motion of a joint, a goniometer is commonly used. Goniometers are instruments that either measure an angle of, for example, a joint of a person, or is an instrument that allows an object to be rotated to a precise angular position. Unfortunately, a patient or athlete is able to accurately use this device on their own. Further, it is sometimes difficult even for clinicians to position and maintain the arms of the goniometer along the bones of the segments of the joint being measure throughout the measurement, and the axis of rotation is not always clear, especially for complex joints.

In another example, wearable clothing including one or more wearable sensors is available to measure a range of motion of a joint. Unfortunately, this clothing requires sophisticated and costly hardware to detect the position of the sensors in the clothing and determine the range of motion based on the position of the sensors.

When performing other physiological measurements, such as measuring changes in volume of a tissue of a person, existing techniques also require sensors to be attached to the skin of the patients and require sophisticated hardware that is large and expensive, or other expensive and sophisticated systems such as ultrasound.

Accordingly, there is a need for systems and methods for performing physiological measurements that do not require the attachment of sensors to the person's body and may be completed by the user themselves without the use of sophisticated hardware or the support of a clinician.

### SUMMARY

In accordance with a broad aspect, a systems and methods of performing a physiological measurement are described herein. The methods include applying a first marker to a portion of a tissue, the first marker having a pattern of markings thereon, each marking of the pattern of markings having an initial position; capturing an image of the pattern of markings at a first time after applying the first marker to the tissue, at least one marking of the pattern of markings having a subsequent position at the first time when the image is captured; determining a change of position of the at least one marking by comparing the initial position of the at least one marking to the subsequent position of the at least one marking; and based on the change of position of the at least one marking, determining the physiological measurement of the tissue.

In at least one embodiment, the physiological measurement is a range of motion of a joint.

In at least one embodiment, the method also include applying a second marker to a fixed reference point of the joint.

In at least one embodiment, applying the first marker to the portion of the tissue includes applying the first marker to one of: an anterior surface of an ankle; a deltoid surface of a shoulder; a lateral surface of a knee; an anterior surface of a knee; a lateral surface of an elbow; and a lateral surface of a hip.

In at least one embodiment, the joint is an ankle and the fixed reference point is a lateral malleolus of a fibula.

In at least one embodiment, the joint is a shoulder joint and the fixed reference point is an acromion of a scapula.

In at least one embodiment, the joint is a knee joint and the fixed reference points are the middle of the patella, the lateral epicondyle of the femur and/or the lateral malleoli of the fibula.

In at least one embodiment, the joint is an elbow joint and the fixed reference point is lateral epicondyle of the humerus.

In at least one embodiment, the joint is a hip joint and the fixed reference point is greater trochanter of the femur.

In at least one embodiment, capturing the image includes capturing a first image of the pattern of markings when the marker is at a first position and capturing a second image of the pattern of markings at a second position a complete spectrum of movement of the joint.

In at least one embodiment, the complete spectrum of movement of the joint includes flexion, abduction and/or rotation of a shoulder.

In at least one embodiment, the complete spectrum of movement of the joint includes flexion, pronation and/or supination of an elbow;

In at least one embodiment, the complete spectrum of movement of the joint includes dorsiflexion, plantarflexion, inversion, eversion, medial rotation and/or lateral rotation of an ankle;

In at least one embodiment, the complete spectrum of movement of the joint includes flexion and/or extension of a knee; and/or

In at least one embodiment, the complete spectrum of movement of the joint includes abduction, adduction and/or extension of a hip.

In at least one embodiment, the physiological measurement is a change in volume of the tissue.

In at least one embodiment the method also includes, prior to applying the marker to the portion of the tissue, applying a stencil to the portion of the tissue, the stencil having the pattern of markings cut out to provide for the semi-permanent ink to pass through openings forming the pattern of markings and into the tissue.

In at least one embodiment, the tissue is adjacent to a surgical incision.

In at least one embodiment, the surgical incision was used during one of: a breast reconstruction or augmentation surgery; a facial plastic or reconstruction surgery; a cardio-thoracic surgery such as a sternal repair after coronary artery bypass graft surgery (CABG); and a hernia surgery.

In at least one embodiment, the pattern of markings includes straight line segment markings and curved line segment markings.

In at least one embodiment, the pattern of markings includes straight line segment markings and curved line segment markings.

In at least one embodiment, the pattern of markings includes a plurality of dots and a plurality of fiducials.

In at least one embodiment, the plurality of fiducials each have a cross shape.

In at least one embodiment, the pattern includes three fiducials, each having a cross shape.

In at least one embodiment, determining the change of position of the at least one marking includes comparing a subsequent position of at least one feature of at least one of the fiducials in the captured image to an initial position of the at least one feature of the of at least one of the fiducials in a template image.

In accordance with another broad aspect, a system for performing a physiological measurement of a tissue of a person is described herein. The system includes a computer processor coupled to a memory, wherein the computer processor is programmed to perform a physiological measurement by: receiving image data from a patient device, the image data including a pattern of markings of a marker on the tissue at a first time after applying the marker to the portion of the tissue; determining a change of position of at least one marking of the pattern of markings by comparing an initial position of the at least one marking to a subsequent position of the at least one marking; based on the change of position of the at least one marking, determining a physiological measurement of the person.

Other aspects and features will become apparent to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is block diagram of a method of performing a physiological measurement, according to at least one embodiment described herein.

FIG. 2A is a side view of a patient's knee shown in extension having a marker thereon, the marker having a pattern of markings according to at least one embodiment described herein.

FIG. 2B is a side view of the patient's knee shown in flexion having the marker having the pattern of markings of FIG. 2A placed thereon.

FIG. 2C is an anterior view of the patient's knee shown in extension having a second marker having the pattern of markings of FIG. 2A placed thereon.

FIG. 3 is a top view of a patient's shoulder shown in flexion, extension and abduction having another marker placed thereon, the marker having the pattern of markings of the marker of FIG. 2A.

FIG. 4A is a top view of a pattern of lines of a marker, according to at least one embodiment described herein, for applying to tissue for performing a physiological measurement.

FIG. 4B is a top view of another pattern of lines of a marker, according to at least one embodiment described herein, for applying to tissue for performing a physiological measurement.

FIG. 4C is a top view of the pattern of lines of FIG. 4B after having undergone lateral movement in response to movement of the underlying tissue.

FIG. 4D is a top view of the pattern of lines of FIG. 4B after having undergone vertical movement in response to movement of the underlying tissue.

FIG. 5A is a patient image showing a marker applied to an injury area, according to at least one embodiment described herein.

FIG. 5B is a template image of a marker 250 in a native or top view of another pattern of lines of a marker, according to at least one embodiment described herein, for applying to tissue for performing a physiological measurement.

FIG. 5C is a top view of the pattern of lines of FIG. 4B after having undergone lateral movement in response to movement of the underlying tissue.

FIG. 6A is a patient image showing a marker applied to an injury area, according to another embodiment described herein.

FIG. 6B is an image of a marker during at least one step of processing a patient image to generate a three-dimensional (3D) image, according to at least one embodiment described herein.

FIG. 6C is a 3D representation of a shape of a target area of a patient generated from a two-dimensional (2D) image, according to at least one embodiment described herein.

FIG. 7A is an image of a marker during at least one step of processing a patient image to generate a 3D image, according to at least one embodiment described herein.

FIG. 7B is an image of a portion of a marker during at least one step of processing a patient image to generate a 3D image, according to at least one embodiment described herein.

FIG. 7C is an image of another portion of a marker during at least one step of processing a patient image to generate a 3D image, according to at least one embodiment described herein.

FIG. 8 is a block diagram of a management system for receiving an image of a tissue and performing a physiological measurement, according to at least one embodiment described herein.

FIG. 9 is a block diagram of a server of the management system for receiving an image of a tissue and performing a physiological measurement, according to at least one embodiment described herein.

### DETAILED DESCRIPTION

Various apparatuses, systems and methods will be described below to provide an example of each claimed embodiment. No embodiment described below limits any claimed embodiment and any claimed embodiment may cover apparatuses, systems and methods that differ from those described below. The claimed embodiments are not limited to apparatuses, systems and methods having all of the features of any one apparatus, system or method described below or to features common to multiple or all of the apparatuses, systems and methods described below.

The embodiments described herein are implemented as apparatuses, systems and methods for performing physiological measurements, such as, without limiting the foregoing, measuring changes in volume of a tissue of a person and/or measuring a range of motion of one or more joints controlled by those tissues, transmitting data related to the physiological measurements to, for example, a medical professional, and processing the data. In at least some embodiments described herein, based on the data, the medical professional can assess, diagnose, recommend and/or provide medical treatment to the patient, and/or communicate with the patient, as necessary.

More specifically, apparatuses, systems and methods for performing physiological measurements including but not limited to the changes in volume of a tissue, and range of motion of joints, are described. The apparatuses, systems and methods provide for a subject (e.g., a person or a patient) to capture an image (e.g. with a camera, a mobile device, etc.) of a part of their body having either a joint of interest or a portion of tissue of interest to perform the physiologic measurement. Prior to capturing the image, a marker is applied to the joint (e.g. on skin covering the joint) or applied to a portion of the tissue of interest (e.g. on skin covering muscle, etc.). These systems, apparatuses and methods may be advantageous for use in rehabilitation settings where they may provide real-time feedback to the patient regarding swelling and/or range of motion of one or more of their joints and thus aid in a determination of the patient's rehabilitation and return to function or sport. They may also help to reduce the amount of time that the patient needs to spend with a physician or rehabilitation specialist.

The marker is an indicia that is used during processing of the image to measure and/or assess movement of tissue and/or a joint underlying the marker. For example, the marker may include a pattern of lines (hereinafter referred to as a marker and/or a reticle or a reticule) positioned thereon that change shape in response to movement of tissue or a joint underlying the marker.

In at least one embodiment, the marker is a sticker, a temporary tattoo, a semi-permanent tattoo or a permanent tattoo.

In at least one embodiment, the marker is a sticker that can be applied to skin of a person.

In at least one embodiment, the marker is a semi-permanent tattoo (i.e. a placement of pigment at least partially under the skin) that has been applied to, or superficial to, the tissue or joint of interest. The semi-permanent tattoo may be applied to skin in a shape that provides one or more reference points for performing the physiological measurement.

Herein, the term “semi-permanent tattoo” is used to refer to the placement or application of pigment, or ink (i.e. a fluid containing pigment) at least partially under the skin. The pigment can be applied directly to the skin by film, or under the skin by a needle, can be applied to a top surface of the skin and allowed to absorb into the skin, can be applied to a top surface of the skin and pushed into the skin (e.g. by compression), can be applied by any combination of the above, or can be applied by any other technique known to the skilled person for applying a semi-permanent tattoo. Herein, the term “semi-permanent tattoo” is contrasted with the term “temporary tattoo”, where the term “temporary tattoo” typically refers to the placement of pigment, or ink, on a top surface of the skin without any, or with minimal, absorption of the pigment or ink into the skin. Typically, semi-permanent tattoos remain on a person's body for a period of time in a range of three days to one year, whereas temporary tattoos remain on a person's body for a period of three days or less.

In at least one embodiment, the marker can be applied by being ink positioned on a film and by being placed directly on the skin by applying pressure against the skin.

In at least one embodiment, the marker can be a sticker held in place on the skin by an adhesive.

The apparatuses, systems and methods described herein provide for the person having at least one marker positioned thereon to capture an image of the at least one marker over various periods of time to perform a physiological measurement at various time points, to thereby assess changes in the physiological measurement over time. The apparatuses, systems and methods described herein provide for a user to capture an image of marker with any camera, such as but not limited to a camera on a mobile device (e.g. a smart phone, a tablet, etc.).

It should also be understood that in some embodiments, the apparatuses, systems and methods described herein provide for the person having the at least one marker positioned thereon to capture a video of the at least one marker for a period of time to perform the physiological measurement and assess changes in the physiological measurement over the time of the video.

As described further below, in these embodiments, a video may be captured showing a range of motion of a joint between a first position and a second position, the motion of the joint between the first position and the second position representing its complete spectrum of movement in a particular direction. For example, the range of motion of the joint between the first position and the second position may represent a complete spectrum of movement of:
flexion, abduction and/or rotation of a shoulder;
flexion, pronation and/or supination of an elbow;
dorsiflexion, plantarflexion, inversion, eversion, medial rotation and/or lateral rotation of an ankle;
flexion and/or extension of a knee; and/or
abduction, adduction, flexion and/or extension of a hip.

It should be understood that as tissue underlying the marker changes shape and/or volume, the pattern of markings on the marker change shape. The change in shape of the pattern of markings on the marker can be used to perform one or more physiological measurements.

For example, the apparatuses, systems and methods described herein may be appropriate for measuring a range of motion of joints such as a shoulder, a knee, an elbow, an ankle and/or a hip.

For example, the change in shape of the pattern of markings on the marker can be used to measure a range of motion of a joint that controls a position of the tissue underlying the marker. In these embodiments, for example, the pattern of markings on the marker may not change shape, but rather may change position relative to a fixed reference point adjacent to or proximate to the marker. In some examples, the fixed reference point may be indicated by a second marker applied to a portion of a tissue of the patient. In some examples, the fixed reference point may be indicated by a second marker applied to a portion of the joint that generally remains stationary during movement of the joint.

For example, the apparatuses, systems and methods described herein may be appropriate for measuring changes in volume of muscle tissue, such as but not limited to the following muscles; biceps, triceps, pectoral, deltoid, quadriceps, hamstrings, calf and anterior muscles of the leg below the knee.

For example, the change in shape of the pattern of markings on the marker can be used to measure a change in bulk volume of the underlying tissue, or position of a joint over time. In these embodiments, for example, the change in shape of the pattern of markings on the marker can indicate a change in surface area of the tissue underlying the marker. The change in surface are of the tissue underlying the marker may indicate swelling and/or atrophy of the underlying tissue.

In at least one embodiment, the apparatuses, systems and methods described herein provide for determining the change in volume of the tissue of interest by measuring, for example, changes in a distance between two or more reference points of the marker over time, changes in curvature of reference lines of the marker over time, etc.

In at least one embodiment, the tissue of interest may be a non-articulating tissue of a patient, such as but not limited to tissues adjacent to surgical incisions as well as surrounding tissue associated with, for example, breast reconstruction or augmentation surgery, facial plastic or reconstruction surgery and/or various cardio-thoracic surgeries including but not limited to sternal repair after coronary artery bypass graft surgery (CABG) and hernia surgeries.

In at least one embodiment, the apparatuses, systems and methods described herein provide for converting a two-dimensional image of the marker into a three-dimensional image of the tissue to determine a change in volume of the tissue.

In at least one embodiment, after capturing the image, the image may be transmitted, for example to a medical professional, for assessment.

In at least one embodiment, the physiological measurements contemplated herein are performed without the use of any sensors or fixtures or other articles other than the markers described herein.

In at least one embodiment, the changes in volume of the tissue may be increases in the volume of the tissue (e.g. tissue swelling).

In at least one embodiment, the changes in volume of the tissue could be decreases in the volume of the tissue (e.g. atrophy of muscle tissue).

For example, the apparatuses, systems and methods described herein may be appropriate for measuring changes in the range of motion of the joints controlled by the above muscles

For example, the systems and methods described herein may be appropriate for monitoring compartment syndrome, such as but not limited to monitoring swelling in a calf or a forearm to monitor for compartment syndrome.

For example, the systems and methods described herein may be appropriate for monitoring changes in volume of fat tissue.

For example, the systems and methods described herein may be appropriate for monitoring temperature change in tissue, such as but not limited to muscle tissue.

For example, the systems and methods described herein may be appropriate for monitoring changes in volume of tissue in response to medication(s).

For example, the systems and methods described herein may be appropriate for monitoring changes in range of motion of joints over a period of time.

For example, the systems and methods described herein may be appropriate for monitoring changes in volume of tissue in response to chronic conditions such as but not limited to heart and kidney failure, where swelling of the lower limbs is common and often worsens with a decline in the underlying condition, and post-operative conditions such as but not limited to patients who have had limb surgery to monitor and inhibit deep venous thrombosis.

For example, the systems and methods described herein may be appropriate for monitoring muscle growth with lifting exercises, such as but not limited to monitoring muscle growth in a biceps to a triceps.

For example, the systems and methods described herein may be appropriate for monitoring distention or weight loss, such as but not limited to monitoring an increase or decrease in tissue volume in an abdomen.

For example, the systems and methods described herein may be appropriate for monitoring respiratory effort, such as but not limited to monitoring an increase or decrease in tissue volume in a chest.

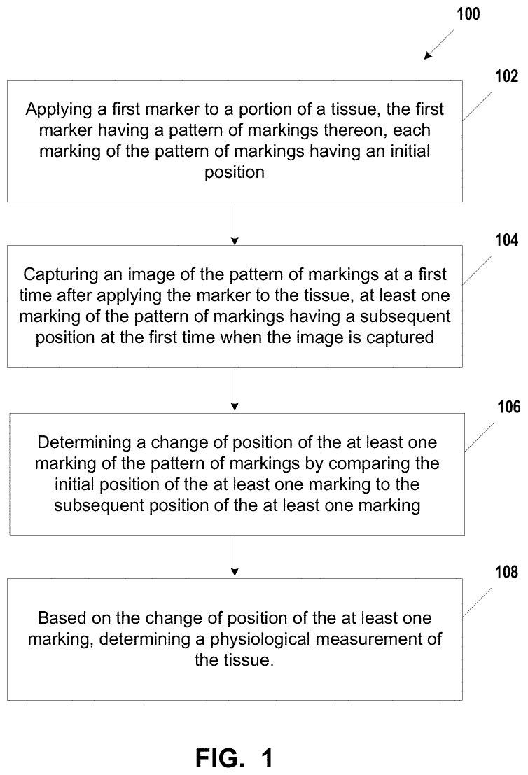

Referring now to FIG. 1, a method 100 of measuring a change in volume of a tissue of a person is shown therein.

At a first step 102, a first marker is applied (i.e. fixed) to a portion of a tissue, such as but not limited to skin of a person. In at least one embodiment, the first marker is applied to the tissue that is being physiologically measured. In at least one embodiment, the marker is applied to tissue (e.g. skin) that is superficial to the tissue (e.g. muscle, fat, etc.) and/or joint being measured and/or monitored.

The marker can be any indicator that can be temporarily or permanently applied to skin of a patient. For example, the marker may be ink that is removable and/or fades from skin over a period of time. Typically, markers appropriate for use in the systems and methods described herein may include an ink that comprises a carrier and a colorant. In at least one embodiment, the carrier may include inks that change colour according to the ambient temperature. The marker may also be a sticker that is applied to the skin and adheres thereon.

In some embodiments, the period of time may be in a range of about one day to six months, or about one month to four months, or of about 90 days.

The marker may be applied to the tissue using any method known to the skilled person for applying temporary markings to skin. For example, the marker may be applied as a sticker onto the skin, the sticker having a pattern of markings thereon.

The marker may be applied as ink directly onto the skin using, for example, a dropper or a pen. The ink may optionally absorb at least partially into the skin. The ink may optionally be applied by spraying the ink onto a stencil or template.

The marker may be injected into the skin using a needle, or a system of needles organized to print the pattern of markings.

In order to ensure that the marker is applied to the skin in a known pattern, first step 102 may include applying a stencil or a template to a portion of the tissue before applying the marker. The stencil may be made of any material appropriate for temporarily adhering to the skin of the person. The stencil or template will have the pattern of the markings cut out of it, so when ink is applied to the stencil it passes through openings of the stencil and absorbs into the skin of the person.

The marker may be applied using a roller of needles arranged in the pattern of the pattern of markings, the needles being small and projecting just far enough into the skin so as to place the ink at the right depth for the length of time required for performing the physiological measurements.

Herein, the pattern of markings applied to the skin may be referred to as a reticle, or reticule, where a reticle is a series or a pattern of fine lines (e.g. straight and/or curved lines).

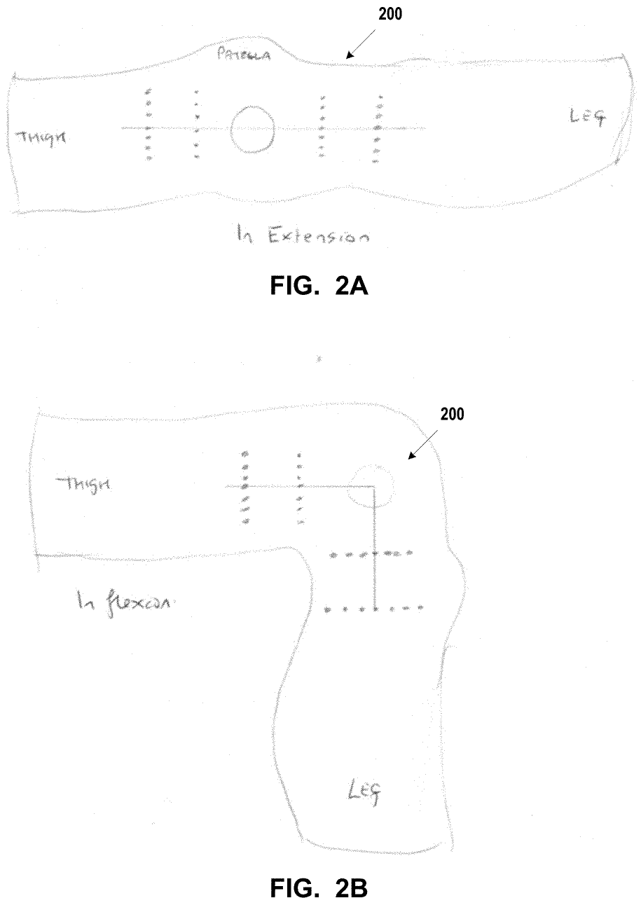

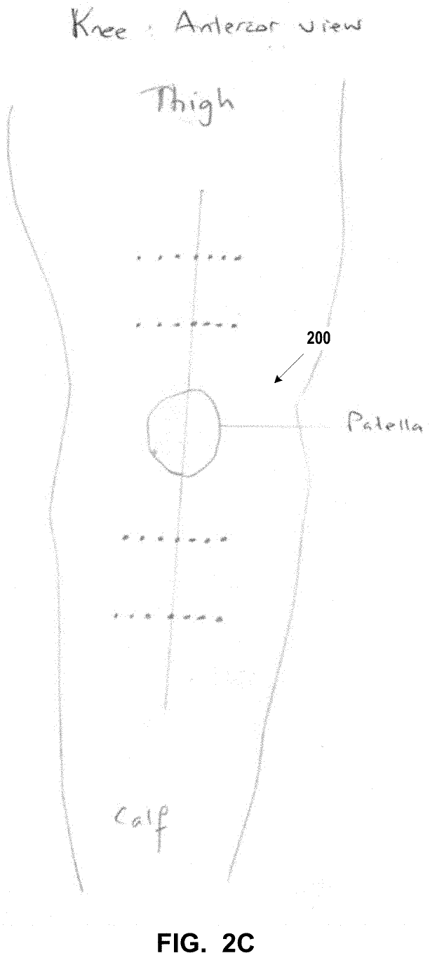

Turning to FIGS. 2A to 2C, illustrated therein is an example of a marking 200 applied to a knee of a patient. In examples where a range of motion of the knee is sought as the physiological measurement, the knee may be positioned in extension (see FIG. 2A), an image of the first marker 200 may be captured, the knee may be positioned in flexion (see FIG. 2B) and a second image of the first marker 200 may be captured. As described above, as the knee moves between being fully extended and fully flexed, first marker 200 will change its shape.

First marker 200 may be positioned on any surface adjacent to or proximate to the joint of interest or on any surface where it is desired to indicate a position relative to another marker or sub-system of markers. In at least one embodiment, the first marker 200 may be placed on an anterior surface of an ankle; a deltoid surface of a shoulder; a lateral surface of a knee; a lateral surface of an elbow; or a lateral surface of a hip.

For example, FIG. 2C shows first marker 200 being positioned on an anterior surface of a knee.

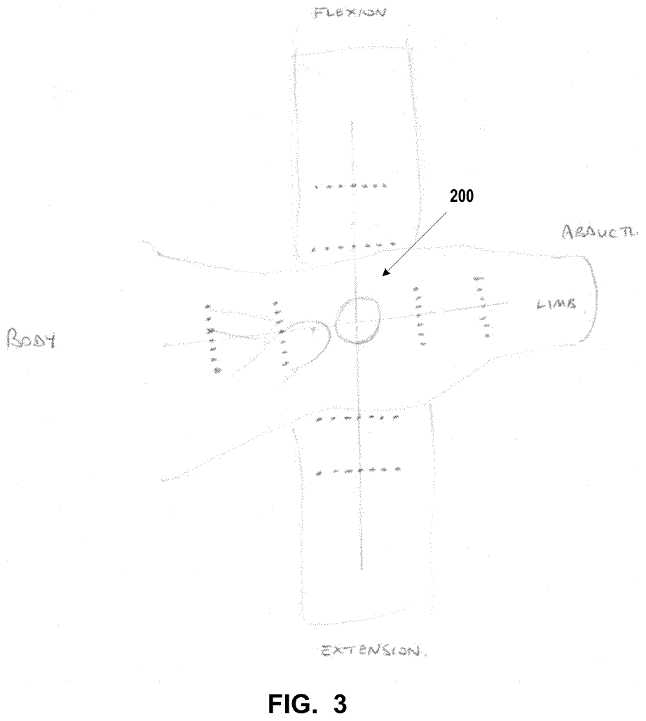

FIG. 3 is a top view of a patient's shoulder shown in flexion, extension and abduction having a first marker 200 placed thereon.

In at least one embodiment, a second marker (not shown) may be provided to the joint to provide a fixed reference point for analysis of the movement and/or change in shape of the marker 200. The second marker may be placed on a part of the joint that generally remains fixed during movement through a range of motion of the joint. For example, the second marker may be placed so as to related to fixed bony prominences of the joint, such as but not limited to, when the joint is a knee, the second marker may be placed on the lateral malleolus of a fibula. For example, when the joint is a shoulder joint, the second marker may be placed on an acromion of a scapula. For example, when the joint is an elbow joint, the second marker may be placed on the lateral epicondyle of the humerus. For example, when the joint is a hip joint, the second marker may be placed on the greater trochanter of the femur.

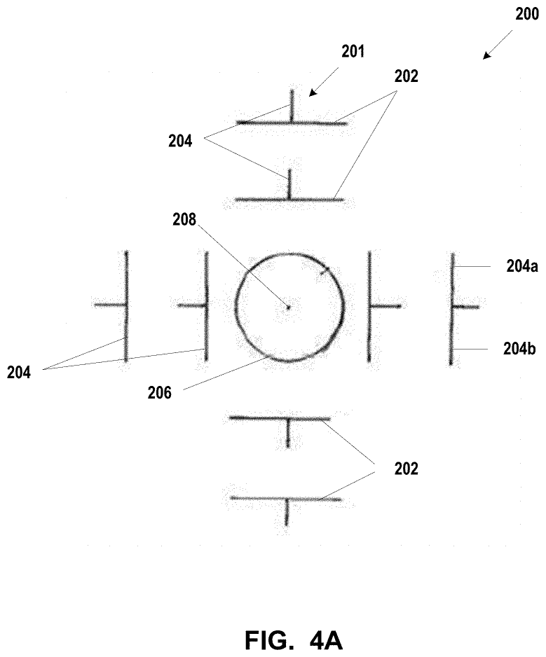

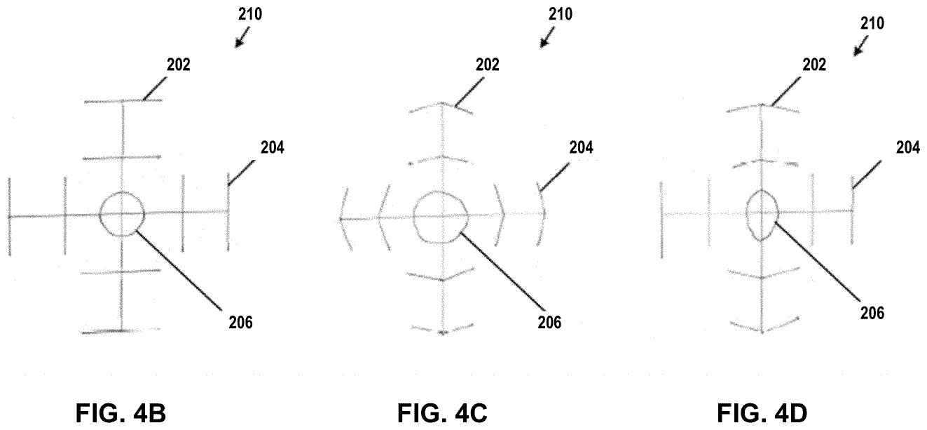

Turning to FIGS. 4A-4D, illustrated therein are examples of markers that may be used with the apparatus, systems and methods described herein.

The initial shape of the marker can provide for measuring a change in volume of a tissue having the marker or, alternatively, of a tissue superficial to the tissue having the marker (e.g. skin).

The marker includes markings that respond to movement of the underlying tissue. By measuring distances between points (e.g. reference points, or reference lines, or straight line segments, or curved line) of the markings shown in the captured image, the image can be used to assess the healing of a joint by measuring both the range of motion of the joint and the state of the muscles as a result of tissue expansion or contraction. The marker may also measure the swelling of the injured joint as well.

Briefly, in one embodiment of a marker shown in FIG. 4A, marker 200 may provide a standardized outcome measure for a medical professional analyzing the tissue. In one embodiment, marker 200 has a plurality of markings 201 including one or more vertical lines 202, horizontal lines 204, curved lines 206 and/or reference points 208. It should be understood that each of the lines 202, 204 also has end points, such as but not limited to end points 204a, 204b shown in FIG. 4A, that can be used as reference points for determining a change in shape of the marker 200 over time.

FIG. 4B shows another example a of markers 210. As shown therein, the one or more vertical lines 202, horizontal lines 204, curved lines 206 and/or reference points 208 may have different arrangements than shown in FIG. 4A. For example, a plurality of one or more vertical lines 202, horizontal lines 204, curved lines 206 and/or reference points 208 may be grouped to form shapes. For example, the one or more vertical lines 202 may be substantially vertical, the one or more horizontal lines 204 may be substantially horizontal, the one or more curved lines 206 may be circular or may form other curved lines, or arc, having other shapes (oblong circles, etc.) and/or the one or more reference points 208 may be positioned at a centre of the marker or, alternatively, may be positioned anywhere within a perimeter of the marker. The perimeter of the marker is an imaginary boundary that is formed by an outermost marking on each of four sides of the marker.

FIG. 4B shows marker 210 comprising vertical lines 202, horizontal lines 204 and a curved line 206 in a neutral configuration. FIG. 4C shows a first stretched configuration of marker 210 where both vertical lines 202 and horizontal lines 204 have been stretched in at least two directions (e.g. the horizontal and the vertical direction) by movement of the underlying tissue. Further, curved line 206 has been stretched.

In FIG. 4D, vertical lines 202 remain unchanged relative to vertical lines 202 in FIG. 4C, however horizontal lines 204 are stretched due to movement of the underlying tissue in one direction (e.g. in a direction perpendicular to the horizontal lines 204 in FIG. 4A). In FIG. 4D, curved line 206 is stretched in another direction due to movement of the underlying tissue in one direction (e.g. in a direction perpendicular to the horizontal lines 204 in FIG. 4A).

It should be understood that the examples of markers shown herein are only a few examples, and the shape and/or configuration and/or arrangement of markings 201 within a marker 200 should be not limited to those arrangements shown in the examples.

Each of the markings 201 of the marker 200 (as well as markers 210, 220, 230) has known dimensions and/or known spacings from other markings within the marker the initial configuration or arrangement of marker 200.

Turning back to FIG. 1, at step 104, an image of the marker on the tissue is captured at a first time after applying the marker to the portion of the tissue. At the first time, at least one marking of the pattern of markings has a subsequent position. In its subsequent position, the at least one marking of the pattern of markings has changed position relative to its first position when applied to the tissue of the person

In embodiments where the physiological measurement is a range of motion of a joint, as the joint moves through its range of motion, a position of at least one marking of the plurality of markings of first marker 200 will change relative to a second marker positioned at a fixed reference point. Accordingly, the position of the first marker 200 can be measured relative to the second marker at the fixed reference point and a range of motion of the joint can be determined.

In embodiments where the physiological measurement is a volume of tissue, as the volume of the tissue, or tissue underlying the tissue having the marker, changes, (e.g. by swelling, atrophy, etc.) a position of at least one marking of the plurality of markings with change within the marker. Accordingly, the dimensions of the markings comprising the marker will change relative to one another (e.g. changes in the length, width and/or spacing between horizontal lines 202, vertical lines 204, curved lines 206 and/or reference points 208). As noted above, the change in dimensions of the markings comprising the marker can be used to measure a change in the volume of the tissue having the marker and/or a tissue superficial to the tissue having the marker.

For example, changes in the length and/or spacing between horizontal lines can indicate swelling in a vertical plane of the tissue, whereas changes in the length and/or spacing of the vertical lines can indicate swelling in a horizontal plane. Similarly, changes in both vertical and horizontal line spacing or distortion can indicate a general state of tissue expansion or contraction. Changes in the physical dimensions of the marker (e.g. changes in spacing, length and/or width of the lines and/or circles of the marker) can be measured by an algorithm of a mobile application (as described further below) when an image of the marker in a subsequent arrangement and/or configuration are captured, such as but not limited to being captured using a camera of a on a smart phone or other mobile device (e.g. tablet).

After an image is captured using the device at step 104, at step 106, a change of position of the at least one marking of the pattern of markings is determined by comparing the initial position of the at least one marking to the subsequent position of the at least one marking dimensions of the marker.

In at least one embodiment, the determining of the change of position of the at least one marking of the pattern of markings can be performed by an application running on a mobile device, such as but not limited to the mobile device having the camera that captured the image.

In another embodiment, the determining of the change of position of the at least one marking of the pattern of markings can be performed by a processor running, for example, on a computer or server that is remote from the mobile device that captured the image of the marker. In this case, prior to determining the change of position of the at least one marking of the pattern of markings, the image captured by the camera of the mobile device can be transmitted from the mobile device to the remote (e.g. lengths of lines and/or symbols of the marker, widths of lines and/or symbols of the marker, spacing between lines and/or symbols of the marker, etc.) in the image to stored dimensions of the first marker 200.

The image capture device can collect and store images of markers for later use.

At a step 108, based on the change of position of each of the markings, the method includes determining a change in volume of the portion of the tissue. After the image is captured and transmitted to a processor for processing, the processor measures the distances between the markings 201 and determines a change in volume of the underlying tissue.

In at least one embodiment, a medical professional may then assess the change in volume of the underlying tissue and, for example, may relate the measured change in volume to an expected outcome from knowledge or from a data base of similar volume changes.

Preferably, the patient can capture images of the marker, or have images captured by a friend, relative, caregiver, etc. on a regular basis, and the medical professional can schedule appointments based on the observed results rather than routine or urgent based upon patient fears.

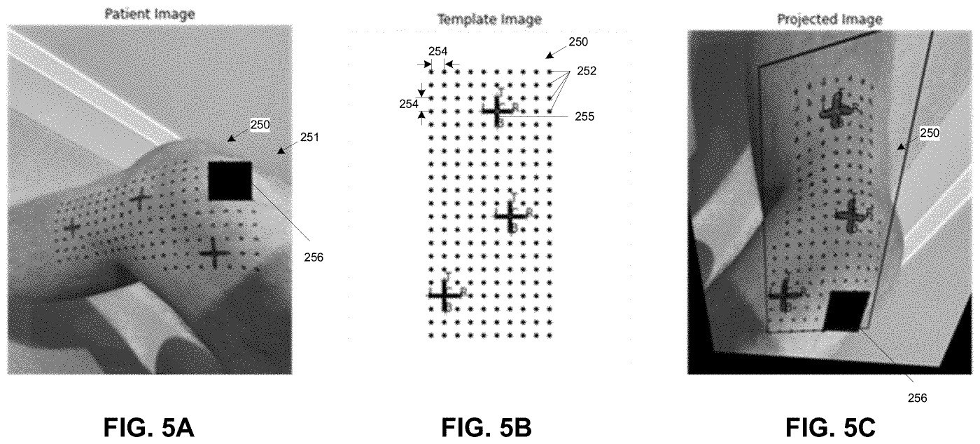

Turning now to FIGS. 5A to 5C, shown in FIG. 5A is a patient image of a limb (e.g., of an injury area or target area 251) of the one or more of the systems described herein. In this embodiment, the user has applied marker 250, as shown in FIG. 5B, to their lower limb.

As noted above, marker 250 of FIG. 5B has a known flattened geometry, meaning that dimensions of the plurality of circular dots 252 each have a known size and a known separation, or spacing 254, from others of the dots 252. Herein, spacing 254 represents a known spacing between any adjacent dots 252 and is not intended to mean that the distances between adjacent dots 252 must be equal. Dots 252 may be spaced apart from each other by unequal distances provided that the distances are known. Marker 250 also includes a plurality of fiducials 255. In at least one embodiment, marker 250 includes at least two fiducials 255. In at least one embodiment, marker 250 includes at least three fiducials 255. In at least one embodiment, marker 250 includes three fiducials 255. In at least one embodiments, marker 250 includes more than three fiducials 255.

In at least one embodiment, each fiducial 255 is used as a point of references and has one or more features that are used to orient and/or determine a size and/or shape of the fiducial. In at least one embodiment, each fiducial 255 may have a same size and/or shape having same features. In at least one embodiment, each fiducial 255 may have a different size and/or shape having different features.

In the embodiment shown in FIGS. 5A to 5C, each fiducial 255 has a cross shape having a top (T), right (R), left (L), and bottom (B) point and a center (C). In this example, one or more of these features may be mapped from the patient image (e.g., FIG. 5A) to a template image (e.g., FIG. 5B).

Each fiducial 255 of the plurality of fiducials embedded within a grid of dots 252 of marker 250 has a known size and known location within marker 250. For example, in at least one embodiment, the fiducials 255 each have a cross shape. It should be understood that the fiducials 255 may have a different shape, including but not limited to a circle, square, rectangle, star, pentagon, hexagon, heptagon, octagon, or the like.

In at least one embodiment, a shape of the fiducials 255 is tracked to recover range of motion of an injury area 251 of a user. In at least one embodiment, fiducials 255 may assist in registration (i.e., association) of template points in a template reference coordinate system (e.g., as defined by the marker 250) to the patient points in the patient image.

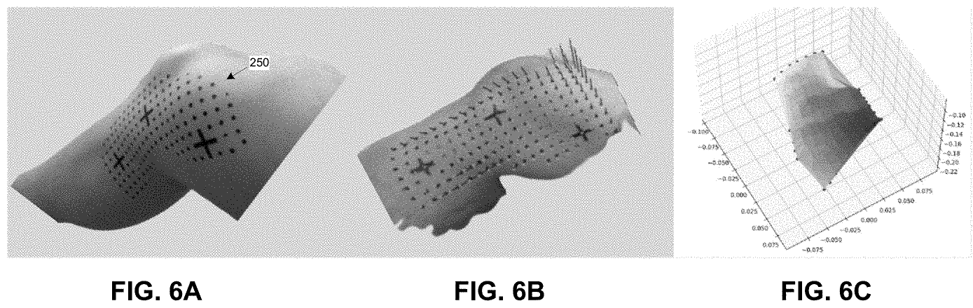

FIG. 6A shows another example of a patient image of one or the methods described herein.

In at least one embodiment, determining a physiological measurement using, for example, marker 250 may include generating a three-dimensional (3D) shape of the injury area 251 based on a two-dimensional (2D) image. In at least one embodiment, the following steps may be performed, inter alia to determine a physiological measurement, such as but not limited to a range of motion of an injury area 251 (e.g., of a joint of a body):
1. Detection of fiducial locations on the patient image (e.g., image shown in FIG. 5A);
2. Association of the template points to the patient image (e.g., associating fiducials 255 of image shown in FIG. 5B to the fiducials 255 of image shown in FIG. 5A);
3. Based on differences of size and/or shape of the fiducials 255 and/or the size and/or shape and/or spacings between dots 252, recovery (e.g., generating) of a 3D shape representing a 3D shape of the tissue underlying marker 250 (see, for example, FIG. 6C); and
4. Tracking of relative 3D shape changes over time.

In at least one embodiment, marker 250 may include a temperature sensor 256 as shown in FIGS. 5A and 5C. In at least one embodiment, temperature sensor 256 may include temperature indicating ink, as described in greater detail below, to provide an indication of a temperature of underlying tissue.

In at least one embodiment, temperature sensor 256 may be used to differentiate fiducials 255 within marker 250 (e.g., to identify fiducials 255 within marker 250), and/or to orient the marker 250 on the person.

Once fiducials 255 have been detected and/or identified in a patient image (e.g., FIG. 5A), in at least one embodiment, features of the fiducials (e.g., top, right, left, bottom and center of each cross) are mapped to a template image (e.g., FIG. 5B). This mapping rotates and/or scales and/or skews the shape of marker 250 from the patient image to match the shape of marker 250 from the template image. FIG. 6B shows an example of marker 250 during at least one step of processing the patient image to generate a 3D image.

In at least one embodiment, a projected image may be provided, for example on a screen of a computing device being used by the user, to provide for the user to align fiducials present on a screen of the computing device with the fiducials 255 of marker 250 present on an injury area 251 to align the fiducials 2550 on the screen with the fiducials on the injury area 251 prior to capturing the patient image.

In at least one embodiment, one or more angles can be derived from fiducials 255 and used to compare an angle present in the patient image to a standard template angle present in the template image, which will determine the real knee ROM measurement. For example, in at least one embodiment, fiducials 255 may be directed to be positioned on known anatomical positions of the injury area and, based on a measured angle between the three of the fiducials 255, an angle of a joint of the target area 251 can be measured.

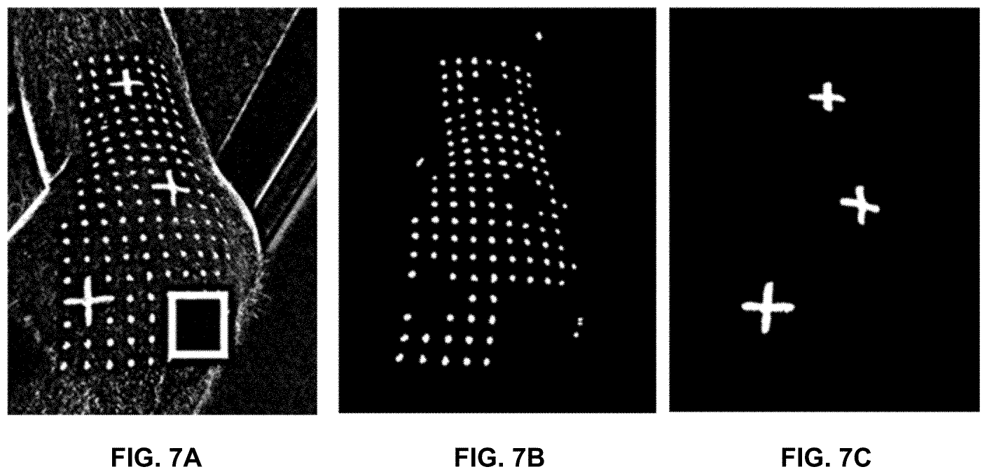

In at least one embodiment, prior to determining the physiological measurement, image processing may be applied to the patient image to isolate the marker 250 from the target area 251. Examples of these images are shown in FIGS. 7A, 7B and 7C.

In at least one embodiment, determining a physiological measurement using, for example, marker 250 may include recovering a solution for a depth and position of each template point as it lies on the 3D surface of the injury area. Herein, template point may refer to any marking in marker 250, including but not limited to dots 252, fiducials 255 (including features thereof) and temperature sensor 256. In at least one embodiment, this is cast as a system of non-linear PDEs that can be simplified by making an assumption that the depth variation on the imaged surface is much smaller than the distance of the surface from the camera (weak-perspective camera) and that local patches on the surface deform rigidly with weak isometry. The depth and normals are recovered for each point on the surface. The volume of the surface can be calculated by integrating the surface normals. The volume can be tracked over time to estimate global changes in swelling for the imaged region. Local changes can also be tracked by visualizing relative motion of grid points with respect to their neighbors over time.

### Temperature-Indicating Ink

In at least one embodiment, the marker that is applied to the tissue may comprise a temperature-indicating ink (e.g., temperature sensor 256).

For example, in some embodiments the temperature-indicating ink may comprise liquid crystals that change colour with changes in temperature of underlying surfaces, such as tissue. These liquid crystals may be Thermochromic Liquid Crystals (TLCs), Cholesteric Liquid Crystals, or the like. In some embodiments, the liquid crystals may be micro-encapsulated for protection, stabilization and to make the liquid crystals easier to use.

In some embodiments, the temperature-indicating ink may provide for analysis of the temperature of the tissue. For example, by analyzing the colour of the ink in the image that is captured, one may be able to assess healing of the tissue based on temperature changes of the tissue.

Management System

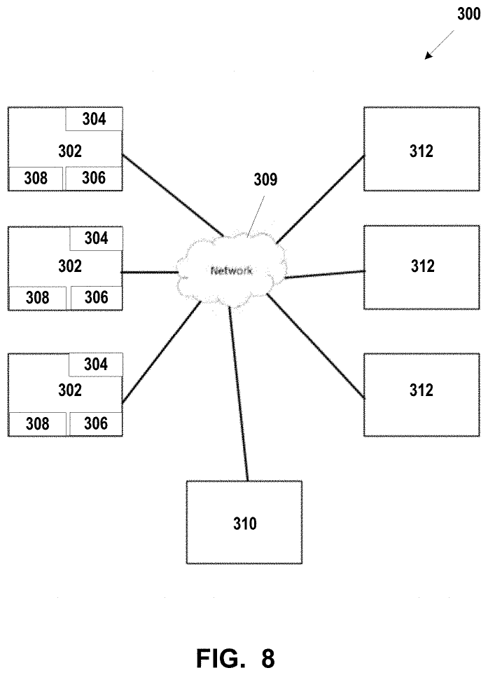

Referring now to FIG. 8, illustrated therein is a system 300 for measuring changed in volume of a tissue. System 300 comprises a plurality of mobile devices 302, each having a camera 304, and a processor 806. For example, a person (e.g. patient) having a tissue can capture a picture of the tissue using their mobile device 302. Stored on the mobile device 302 is an application 308. The person can use the mobile application 308 to send the picture to server 310 (via network 309) for redistribution to a physician device 312 for assessment. Medical professional device 312 can send information back to server 310 or directly to mobile device 302.

The server 310 may communicate with the plurality of mobile devices 302, a plurality of physician devices 312 via network 309. The server 310 may be a purpose built machine designed specifically for implementing a system and method for creating maps of a facility.

The server 310, patient devices 302 and physician devices 312 may be a server computer, desktop computer, notebook computer, tablet, PDA, smartphone, or another computing device. The devices 302, 310 and 312 may include a connection with the network 309 such as a wired or wireless connection to the Internet. In some cases, the network 309 may include other types of computer or telecommunication networks. The devices 302, 310 and 312 may include one or more of a memory, a secondary storage device, a processor, an input device, a display device, and an output device. Memory may include random access memory (RAM) or similar types of memory. Also, memory may store one or more applications for execution by processor. Applications may correspond with software modules comprising computer executable instructions to perform processing for the functions described below. Secondary storage device may include a hard disk drive, floppy disk drive, CD drive, DVD drive, Blu-ray drive, or other types of non-volatile data storage. Processor may execute applications, computer readable instructions or programs. The applications, computer readable instructions or programs may be stored in memory or in secondary storage, or may be received from the Internet or other network 309. Input device may include any device for entering information into device 302, 310 and 312. For example, input device may be a keyboard, keypad, cursor-control device, touch-screen, camera, or microphone. Display device may include any type of device for presenting visual information. For example, display device may be a computer monitor, a flat-screen display, a projector or a display panel. Output device may include any type of device for presenting a hard copy of information, such as a printer for example. Output device may also include other types of output devices such as speakers, for example. In some cases, devices 302, 310 and 312 may include multiple of any one or more of processors, applications, software modules, second storage devices, network connections, input devices, output devices, and display devices.

Although devices 302, 310 and 312 are described with various components, one skilled in the art will appreciate that the devices 302, 310 and 312 may in some cases contain fewer, additional or different components. In addition, although aspects of an implementation of the devices 302, 310 and 312 may be described as being stored in memory, one skilled in the art will appreciate that these aspects can also be stored on or read from other types of computer program products or computer-readable media, such as secondary storage devices, including hard disks, floppy disks, CDs, or DVDs; a carrier wave from the Internet or other network; or other forms of RAM or ROM. The computer-readable media may include instructions for controlling the devices 302, 310 and 312 and/or processor to perform a particular method.

In the present description, devices 302, 310 and 312 are described performing certain acts. It will be appreciated that any one or more of these devices may perform an act automatically or in response to an interaction by a user of that device. That is, the user of the device may manipulate one or more input devices (e.g. a touchscreen, a mouse, or a button) causing the device to perform the described act. In many cases, this aspect may not be described below, but it will be understood.

As an example, it is described below that the devices 302, 310 and 312 may send information to each other. For example, a person user may manipulate one or more input devices (e.g. a mouse, touchscreen, a keyboard, etc.) to interact with a user interface displayed on a display of the patient device 02. Generally, the device 302 may receive a user interface from the network 309 (e.g. in the form of a webpage). Alternatively or in addition, a user interface may be stored locally at a patient device 302 (e.g. a cache of a webpage or a mobile application).

Server 310 may be configured to receive a plurality of information, from each of the plurality of patient devices 302 and physician devices 310. Generally, the information may comprise at least an identifier identifying the patient or physician, respectively. For example, the information may comprise one or more of a username, e-mail address, password, or social media handle. The server may also receive an image of a tissue for devices 302, as described above, for a physician to assess the physiological measurement and/or movement.

In response to receiving information, the server 310 may store the information in a storage database. The storage database may correspond with secondary storage of the devices 302, 312. Generally, the storage database may be any suitable storage device such as a hard disk drive, a solid state drive, a memory card, or a disk (e.g. CD, DVD, or Blu-ray etc.). Also, the storage database may be locally connected with server 806. In some cases, the storage database may be located remotely from server 310 and accessible to server 310 across a network for example. In some cases, storage database may comprise one or more storage devices located at a networked cloud storage provider.

Patient device(s) 302 may be associated with a patient account. Similarly, the physician device 312 may be associated with a physician account. Any suitable mechanism for associating a device with an account is expressly contemplated. In some cases, a device may be associated with an account by sending credentials (e.g. a cookie, login, or password etc.) to the server 310. The server 310 may verify the credentials (e.g. determine that the received password matches a password associated with the account). If a device is associated with an account, the server 806 may consider further acts by that device to be associated with that account.

Devices 302, 312 and server 310 may communicate asynchronously, for example, by using an implementation of the WebSocket protocol, such as Socket.IO. Updates may be sent from the server 310 to each of the devices 302, 312 in real time as interrupts, i.e., without polling. Likewise, user interaction data may be sent from each of the devices 302, 312 to the server 310 in real time as interrupts, i.e., without polling.

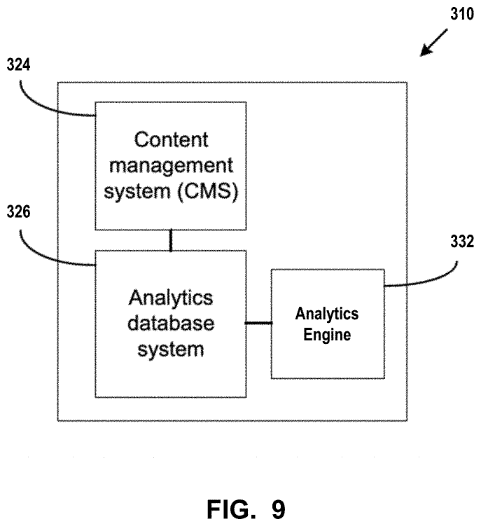

Turning now to FIG. 9, illustrated therein is the server 310, in accordance with an embodiment. The server 310 includes a content management system (CMS) 324, and an analytics database system 326. The server 310 may include multiple backend devices, e.g., servers. The CMS 324 and the analytics database system 326 may be hosted by the server 310.

In some embodiments, the CMS 324 may be a frontend interface application, typically, implemented as a web service. In some embodiments, CMS 324 stores content, including information relating to the changes of either or both swelling and range of motion in the injured area, handles updates to the content received from the devices 302, 312 and provides content to the devices 302, 312. For example, the CMS 324 may be a no structured query language (NoSQL) database application.

In some embodiments, the analytics database system 326 includes or is operatively connected to an analytics engine 332. The analytics database system 826 may be a database application, typically implemented as a web service. The analytics database system 326 stores all user interactions, e.g., user selections or “hits”, searches, dates, types of mobile device, and generates analytics relating to the user interactions. Advantageously, because user interactions are recorded for several different devices of the devices 302, 312 a relatively large sample size is obtained. Further, the analytics database system 326, with analytics engine 332, performs and stores aggregate data analysis on the collective data set that can allow for analysis of all parameters relating to the subject such as range of motion, swelling of joint or tissues around the joint, institutional treatment outcomes, individual surgeon and provider outcomes and any other appropriate opportunities for analysis of different types of treatment options.

In some embodiments, the application 308, when launched on the mobile device 302, takes control of the camera 304. In some embodiments, an identity of the patient is confirmed prior to launching the application. For example, the application 308 can confirm the identity of the patient using one or more of facial recognition software, an iris scan or a fingerprint scan on the mobile device 302. Confirmation information generated based on one of the aforementioned techniques of confirming an identity can be recorded and linked to the medical record at server 310. Confirmation can occur when the application 308 has been downloaded into mobile device 302. In another example, a physician can provide confirmation information through physician device 312 such as by entering a license number, for example. Physician confirmation information cannot be viewed or retrieved by the patient and but resides on server 310.

When application 308 is launched and camera 304 is focused on the tissue, application 308 may assume control by the camera 304 through recognition of, for example, specific markers of marker 200. The application 308 can then project an azimuth onto a display of the mobile device 302 which can guide the holder of the device 302 to align the camera 304 with the marker 200 to the most optimal position to get a representative picture of the marker 200 and/or the tissue.

Application 308 can use the marker 200 to both guide the device 302 as to positioning and to measure changes in volume of the tissue.

In another embodiment, the patient is able to control who has access to the image date transferred via application 308, so that various medical professionals can have access to the image data. Access to data can be configured at server 310 where the image data is stored. In some embodiments, image data may not be stored on the device 302. Rather, a patient can request to receive image data stored on server 310 for a period of time after providing the image data to server 310 (e.g. for a period of 2 years after the service is used).

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

What is claimed is:

1. A method of performing a physiological measurement, the method comprising:
applying a first marker to a portion of a tissue, the first marker having a pattern of markings thereon, each marking of the pattern of markings having an initial position;
capturing an image of the pattern of markings at a first time after applying the first marker to the tissue, at least one marking of the pattern of markings having a subsequent position at the first time when the image is captured;
determining a change of position of the at least one marking by comparing the subsequent position of the at least one marking in the captured image to the initial position of the at least one marking; and
based on the change of position of the at least one marking, determining the physiological measurement of the tissue.

2. The method of claim 1, wherein the physiological measurement is a range of motion of a joint.

3. The method of claim 2 further comprising applying a second marker to a fixed reference point of the joint.

4. The method of claim 3, wherein applying the first marker to the portion of the tissue includes applying the first marker to one of:
an anterior surface of an ankle;
a deltoid surface of a shoulder;
a lateral surface of a knee;
an anterior surface of a knee;
a lateral surface of an elbow; and
a lateral surface of a hip.

5. The method claim 2, wherein capturing the image includes capturing a first image of the pattern of markings when the marker is at a first position and capturing a second image of the pattern of markings at a second position to determine a complete spectrum of movement of the joint.

6. The method of claim 5, wherein the complete spectrum of movement of the joint includes flexion, abduction and/or rotation of a shoulder.

7. The method of claim 5, wherein the complete spectrum of movement of the joint includes flexion, pronation and/or supination of an elbow.

8. The method of claim 5, wherein the complete spectrum of movement of the joint includes dorsiflexion, plantarflexion, inversion, eversion, medial rotation and/or lateral rotation of an ankle.

9. The method of claim 5, wherein the complete spectrum of movement of the joint includes flexion and/or extension of a knee.

10. The method of claim 5, wherein the complete spectrum of movement of the joint includes abduction, adduction and/or extension of a hip.

11. The method of claim 1, wherein the physiological measurement is a change in volume of the tissue.

12. The method of claim 1 further comprising, prior to applying the marker to the portion of the tissue, applying a stencil to the portion of the tissue, the stencil having the pattern of markings cut out to provide for the semi-permanent ink to pass through openings forming the pattern of markings and into the tissue.

13. The method of claim 12, wherein the tissue is adjacent to a surgical incision.

14. The method of claim 13, wherein the surgical incision was used during one of:
a breast reconstruction or augmentation surgery;
a facial plastic or reconstruction surgery;
a cardio-thoracic surgery such as a sternal repair after coronary artery bypass graft surgery (CABG); and
a hernia surgery.

15. The method of claim 1, wherein the pattern of markings includes straight line segment markings and curved line segment markings.

16. The method of claim 1, wherein the pattern of markings includes a plurality of dots and a plurality of fiducials.

17. The method of claim 16, wherein the plurality of fiducials each have a cross shape.

18. The method of claim 16, wherein the pattern includes three fiducials, each having a cross shape.

19. The method of claim 16, wherein determining the change of position of the at least one marking includes comparing a subsequent position of at least one feature of at least one of the fiducials in the captured image to an initial position of the at least one feature of the of at least one of the fiducials in a template image.

20. A system for performing a physiological measurement of a tissue of a person, the system comprising:
a computer processor coupled to a memory, wherein the computer processor is programmed to perform a physiological measurement by:
receiving image data from a patient device, the image data including a pattern of markings of a marker on the tissue at a first time after applying the marker to the portion of the tissue;
determining a change of position of at least one marking of the pattern of markings by comparing a subsequent position of the at least one marking to an initial position of the at least one marking; and
based on the change of position of the at least one marking, determining a physiological measurement of the person.
